(19) United States
(12) Patent Application Publication
(10) Pub. No.: US 2002/0061571 A1
(43) Pub. Date: May 23, 2002

(54) Novel isoform of myotonic dystrophy associated protein kinase and uses thereof

(76) Inventors: Mani S. Mahadevan, Madison, WI (US); Gustavo Tiscornia, Madison, WI (US)

Correspondence Address: SCHWEGMAN, LUNDBERG, WOESSNER & KLUTH, P.A., P.O. BOX 2938, MINNEAPOLIS, MN, 55402, US

(21) Appl. No.: 09/813,289

(22) Filed: Mar. 20, 2001

Related U.S. Application Data
(60) Provisional application No. 60/190,590, filed on Mar. 20, 2000.

(51) Int. Cl. C12N 9/12; C12Q 1/68; G01N 33/567; C07H 21/04

(52) U.S. Cl. 435/194; 435/6; 435/7.21; 435/325; 435/320.1; 536/23.2

(57) ABSTRACT

The invention provides a novel DMPK isoform, isolated and purified RNA encoding the novel isoform, and methods of detecting the novel isoform, e.g., to predict disease outcome or detect disease.

### PRIORITY OF INVENTION

This application claims priority from U.S. provisional application No. 60/190,590, filed Mar. 20, 2000.

### STATEMENT OF GOVERNMENT RIGHTS

This invention was made, at least in part, with a grant from the Government of the United States (grant 1-ROI-AR45992-01 from the National Institutes of Health). The Government may have certain rights to the invention.

### BACKGROUND OF THE INVENTION

Myotonic dystrophy (DM) is an autosomal dominant inherited neuromuscular disorder with a global incidence of 1 per 8000 (Harper, 1989). There are two distinct forms, an adult onset and a congenital form of DM. Adult onset DM is primarily characterized by myotonia, muscle weakness and wasting. However, it also affects a number of organ systems resulting in cataracts, cardiac conduction abnormalities, testicular atrophy, male pattern baldness and insulin resistance. Hypotonia, mental retardation, delayed muscle maturation and developmental abnormalities characterize congenital DM, the most severe form of the disease.

The DM mutation was identified as an expansion of a CTG triplet repeat in the 3′ untranslated region (3′UTR) of a gene encoding a serine-threonine protein kinase (DMPK) (Brook et al., 1992; Fu et al., 1992; Mahadevan et al., 1992). The DM mutation was one of the first “triplet repeat” mutations identified. Although it has been eight years since its discovery, the DM mutation is one of the most notable enigmas in human genetics. Given the location of the (CTG)ₙ in the 3′ untranslated region of the DM protein kinase (DMPK) gene, it has been difficult to reconcile the dominant nature of the DM mutation (a single copy causes a phenotype in carrying individuals).

The CTG tract, normally between 5 and 37 triplets, displays meiotic and mitotic instability (Mahadevan et al., 1992; Tsilfidis et al., 1992) when it reaches a length of about 50 repeats, and can expand up to several thousand repeats when transmitted to subsequent generations. Thus, the mutation is what is known as a “dynamic mutation” whereby children inherit a gene that is not exactly the same as the parent's gene. Though clinical studies have shown a general positive correlation between the length of the repeat tract and disease severity (Hunter et al., 1992; Redman et al., 1993), there is considerable overlap among patients with respect to genotype/phenotype correlation. That is, there are individuals with a large number of repeats who have very few clinical symptoms and individuals with the same number of repeats that have very severe clinical symptoms.

The function of DMPK and the mechanisms by which the DM mutation causes disease are unknown. One proposed mechanism for DM pathogenesis is a defect in RNA metabolism (Groenen and Wieringa, 1998). It has been suggested that the mutation affects processing and/or transport of DMPK mRNA (Fu et al., 1993; Jansen et al., 1992; Krahe et al., 1995; Sabourin et al., 1993), and perhaps other mRNAs sharing the pathway (Morrone et al., 1997; Timchenko et al., 1996; Wang et al., 1995). Moreover, several groups have reported a negative effect on DMPK expression (Carango et al., 1993; Fu et al., 1993; Hofmann-Radvanyi et al., 1993; Krahe et al., 1995). Others have demonstrated by northern blotting (Davis et al., 1997), RT-PCR (Hamshere et al., 1997) and RNA-fluorescent in situ hybridization that the mutant DMPK transcript is retained within the nucleus and forms distinct RNA foci (Taneja et al., 1995). Recently, a cell culture model was described in Amack et al. (1999) in which the expression of a mutant DMPK 3′UTR mRNA, as part of a chimeric transcript, decreased protein production through nuclear retention of the mRNA and RNA foci formation. These results are consistent with a model in which haploinsufficiency of DMPK could be a pathogenic mechanism in DM. In support of this, phenotypic analysis of heterozygote and homozygote DMPK knockout mice has demonstrated a cardiac conduction defect similar to that seen in about 70% of DM patients (Berul et al., 1999). Although this suggests that haploinsufficiency of DMPK contributes to at least this aspect of the disease, many of the cardinal features of DM including myotonia, cataracts, and significant muscle wasting are absent in these mice (Jansen et al., 1996; Reddy et al., 1996). Furthermore, these symptoms are also absent in mice overexpressing a normal human DMPK transgene (Jansen et al., 1996). Thus, these results suggest that a simple dosage effect on DMPK does not account for all the clinical features of DM.

One mechanism proposed to play a role in DM pathogenesis is a trans effect at the RNA level (Morrone et al., 1997; Philips et al., 1998; Wang et al., 1995). Indeed, in the cell culture model of Amack et al. (1999), expression of a mutant (but not wild type) DMPK 3 ′UTR mRNA resulted in inhibition of myoblast differentiation (Amack et al., 1999), a phenotype very similar to the histopathology found in muscle biopsies from congenital DM patients (Sarnat and Silbert, 1976). This could occur through altered interactions with RNA-binding proteins. Several groups have identified proteins capable of binding to CUG motifs (Bhagwati et al., 1996; Timchenko et al., 1996); one such protein, CUG-BP has been implicated in a trans effect (Philips et al., 1998). However, the effect on DMPK expression of proteins which bind to regions other than the CUG motif in the 3′UTR of DMPK has not been reported.

Thus, what is needed is a method to detect RNA-binding proteins that interact with non-CUG regions or higher order structures of the DMPK 3 ′UTR which could be involved in RNA mediated DM pathogenesis. What is also need is a method to detect a marker associated with the severity or incidence of DMPK-associated disorders.

### SUMMARY OF THE INVENTION

The invention provides an isolated and purified polypeptide or peptide which comprises an amino acid sequence encoded by exon 16 of the DMPK gene, and isolated and purified nucleic acid, e.g., mRNA or cDNA, which encodes such a polypeptide or peptide. As described hereinbelow, at least six RNA-binding proteins interact with the DMPK 3′UTR. Four of these proteins were identified as hnRNP C, polypyrimidine binding protein (PTB), and the splicing factors U2 auxiliary factor (U2AF) and PTB associated splicing factor (PSF). Their binding sites were mapped to two specific sequences 3′ of the CUG tract. Careful examination of the sequences adjacent to one of the binding sites revealed a potential 3′ splice site, 34 bases 3′ to the CUG repeats. Functional analyses showed that this splice junction was utilized and its use yielded a novel isoform of DMPK in which the CUG repeats were excluded from the mRNA and which encoded a new carboxy terminus (SEQ ID NO:2 encoded by SEQ ID NO:1). In contrast to (CUG)ₙ containing mRNAs, the novel isoform is not retained in the nucleus in DM cells, resulting in imbalances in relative levels of cytoplasmic DMPK mRNA isoforms. In addition, the CUG repeats were found to be essential for the function of the 3′ splice site. Using DM patient material, this splice site was shown to be functional even in the presence of large CUG expansions. Moreover, the novel mRNA isoform was not subject to the nuclear retention suffered by DMPK mRNA isoforms containing CUG expansions resulting in imbalance of cytoplasmic forms of DMPK mRNA isoforms.

Thus, the DM mutation is formally in the last intron of the gene. Further, the CUG repeats are necessary for splicing at a splice site 3′ to the repeat, indicating that the CUG repeats are cis acting elements. The presence of a novel isoform of DMPK mRNA that is not susceptible to nuclear entrapment (due to the absence of an expanded CUG tract) may yield an alteration in the relative levels of different DMPK isoforms. Given evidence that DMPK forms multimers, an alteration in the relative levels of DMPK isoforms and the concomitant changes in the composition of DMPK complexes could have dominant effect.

The invention also provides a fusion polypeptide or peptide comprising an amino acid sequence encoded by exon 16 of the DMPK gene. The polypeptide, peptide, fusion polypeptide or fusion peptide, e.g., a fusion peptide comprising glutathione S transferase (GST) sequences linked to sequences including SEQ ID NO:2, of the invention is useful to prepare antibodies that specifically bind to the novel DMPK isoform. To determine the relative ratio of the difference DMPK isoforms, the antibody of the invention and antibodies that recognize other DMPK isoforms may be employed. For example, a fusion polypeptide comprising GST and an amino acid sequence encoded by exons 9-15 of the DMPK gene was prepared and employed to prepare antisera that recognizes all DMPK isoforms. The determination of DMPK isoform ratios may be useful to predict the severity of symptoms in DM patients, or to determine predisposition to DM. A peptide of the invention is also useful as an epitope tag in non-human cells, tissues or organisms, as the peptide sequences encoded by exon 16 may be unique to human. For example, an antibody that was raised to a fusion peptide of the invention, e.g., a fusion comprising SEQ ID NO:2, did not bind to non-DMPK transgenic mice and so is useful to detect expression of recombinant fusion proteins in mice.

Another strategy has been to generate peptides that are conjugated to KLH, wherein the DMPK peptides are about 20-25 amino acids. Two peptides sequences that can be used, for example, are:

AEALTWMGKLQAWEGSKPGRPC (SEQ ID NO:14); and

SILHAPPPIVGSQSAKLSCA (SEQ If) NO:15).

These peptides have been used as antigens to generate polyclonal antibodies specific to E16 in rabbits and rats, as well as in mouse. However, any peptide sequence comprising the amino acid sequences from E16 could be used to generate E16 specific antibodies. These antibodies can be used in conjunction with an antibody that detects all forms of DMPK in order to determine the effect of the DM mutation on DMPK protein isoforms.

The invention also provides an isolated and purified antibody, e.g., monoclonal or polyclonal antibodies, specific for the novel isoform of DMPK, e.g., the antibody specifically recognizes the carboxy terminus of the novel isoform. For example, rabbits are immunized with a peptide comprising SEQ ID NO:2 or an immunogenic portion thereof, or a fusion peptide comprising SEQ ID NO:2, and polyclonal antisera specific for the novel isoform isolated. Alternatively, spleen cells from immunized animals are fused to myeloma cells to produce hybridomas. The hybridomas are then screened to identify ones secreting a monoclonal antibody specific for a polypeptide or peptide comprising the carboxy terminal sequences of the novel DMPK isoform. These antibodies are useful to detect the novel DMPK isoform in biological samples, e.g., clinical samples, to detect the relative amount of the novel isoform to other isoforms. For example, the antibodies of the invention and those which recognize exon 16 or other exons, e.g., exons 9-15, may also be useful to detect muscle damage, e.g., such as damage during or subsequent to myocardial infarction. In particular, when cells die, the cellular contents are released to the bloodstream, causing the levels of molecules in the bloodstream, such as DMPK, to increase rapidly. Thus, early detection of an increase in levels of DMPK, e.g., in serum, preferably within 6 hours or less, e.g., 2 hours after myocardial infarction, would allow a rapid diagnosis of an ongoing myocardial infarction and/or other muscle damage.

The invention further provides a method to detect a particular isoform of DMPK. The method comprises contacting a sample of nucleic acid obtained from a biological sample, e.g., mRNA or cDNA, with at least one primer specific for an isoform of DMPK RNA which includes exon 16 under conditions effective to amplify at least a portion of DMPK nucleic acid so as to yield amplified DMPK nucleic acid. Then the presence, absence or amount of the isoform of DMPK nucleic acid which includes exon 16 is detected or determined. In another embodiment of the invention, the relative amount of at least two isoforms of DMPK is detected or determined. Thus, a plurality of samples is each contacted with at least one primer specific for one isoform of DMPK RNA under conditions effective to amplify DMPK nucleic acid so as to yield amplified DMPK nucleic acid. Alternatively, a single sample is contacted with a plurality of primers, wherein at least one primer that is specific for each isoform is present. The presence, absence or amount of each isoform of DMPK is detected and the relative amount of the isoforms determined. Preferably, the size of the amplified product correlates to the present and/or amount of a specific isoform in the sample.

Also provided is a method to detect an isoform of DMPK. The method comprises contacting a biological sample from a mammal with an antibody of the invention so as to form a complex. Complex formation is then detected or determined. Such a method, or one which detects the relative amounts of all, or a certain subset of, DMPK isoforms, may be useful to detect a mammal having, or at risk of, a DMPK-associated disease, e.g., myotonic dystrophy, or other disorders or conditions characterized by muscle damage, e.g., myocardial infarction. Hence, the alteration in relative levels of DMPK compared to the relative levels in normal individuals can be employed to correlate to predisposition to disease or to disease severity.

Thus, the invention also provides a diagnostic method. The method comprises detecting or determining in a physiological sample from a mammal at risk of, or having, a condition associated with muscle damage or dysfunction the amount of a DMPK RNA comprising DMPK exon 16 and correlating the amount to muscle damage or dysfunction. Further provided is a diagnostic method in which the relative amount of RNA encoding at least two isoforms of DMPK is detected or determined and employed to correlate the amount to muscle damage or dysfunction. Preferably, at least one isoform is encoded by RNA comprising DMPK exon 16.

Also provided is a diagnostic method comprising detecting or determining in a physiological sample from a mammal at risk of, or having, a condition associated with muscle damage or dysfunction the amount of a DMPK isoform encoded by RNA comprising DMPK exon 16 and correlating the amount to muscle damage or dysfunction. Preferably, the antibody of the invention is employed to detect the amount of a DMPK isoform encoded by RNA comprising DMPK exon 16. In another embodiment, the relative amount of at least two DMPK isoforms is detected or determined and the amount correlated to muscle damage or dysfunction. Physiological samples include, but are not limited to fluid samples, e.g., blood, or tissue samples, e.g., muscle such as skeletal or heart muscle, brain or testes.

The invention further provides kits useful to detect the novel DMPK isoform. Thus, the invention provides a diagnostic kit for detecting or determining a DMPK isoform which comprises packaging, containing, separately packaged, a solid phase which binds a capture antibody; and a known amount of a detection antibody which specifically binds to a DMPK isoform encoded by RNA comprising DMPK exon 16. In yet another embodiment, the invention provides a diagnostic kit for detecting or determining the ratio of DMPK isoforms which comprises packaging, containing, separately packaged, an antibody which specifically binds to a DMPK isoform encoded by RNA comprising DMPK exon 16; and an antibody which detects isoforms of DMPK other than one encoded by RNA comprising DMPK exon 16. Thus, the invention provides a diagnostic kit comprising an antibody which recognizes the novel DMPK isoform, as well as an antibody which recognizes all isoforms, e.g., an antibody that binds to any one of exons 9-15.

Also provided is a transgenic mouse, the genome of which comprises a construct comprising a human DMPK gene. For example, the construct may comprise genomic human DMPK sequences up to exon 5 and DMPK cDNA sequences for exons 5-16, or any combination the genome of which thereof. In particular, one line of transgenic mice was prepared which comprises a construct comprising 5′ sequences of the genomic human DMPK gene up to exon 5 and cDNA from exons 5-14 and 16, and another line was prepared which comprises a construct comprising 5′ sequences of the genomic human DMPK gene up to exon 5 and cDNA from exons 5-15. In this embodiment, an endogenous DMPK promoter expresses the encoded DMPK isoforms. In another embodiment, a tetracycline inducible operator is employed in the construct so that DMPK expression in the transgenic mouse is inducible.

### BRIEF DESCRIPTION OF THE FIGURES

FIG. 1. Detection of RNA-binding proteins interacting with the DMPK 3′UTR. (A) UV-crosslinking of a DMPK 3′UTR (CUG)₅ riboprobe with protein extracts (nuclear extract (NE), cytoplasmic extract (CE)) from HeLa and C2C12 detects specific protein complexes. Extracts in lanes 1 and 2 have been treated with proteinase K prior to riboprobe incubation. Nuclear extracts from both cell lines show conserved complexes at 43, 60 and 120 kDa with other bands at 50 and 75 kDa. p50 is more obvious in cytoplasmic extracts. (B) Competition with 400×molar excess of unlabeled probes reveals specificity of RNA-protein interactions. Competitor riboprobes (lanes 2-5) are indicated. Only the DMPK 3′UTR RNA (lane 2) competes effectively for binding. A (CUG)₁₀ oligonucleotide (lane 5) effectively binds the two lower bands of the p60 complex, with no effect on other interactions. The corresponding DNA probes (lanes 6, 7) are ineffective competitors.

FIG. 2. RNA-binding proteins interact with two distinct sequences 3′ to CUG repeats. (A) UV-crosslinking of HeLa nuclear protein extracts with riboprobes from various regions of the DMPK 3′UTR shows that p43, p60 and p120 proteins interact with sequences 3′ of the CUG repeats (dwn) but not with sequences 5′ of the repeats (up) or the (CUG) repeats (up+(CUG)₅₇). (B) Riboprobes from 5 sections (a-e) of the downstream region (dwn) revealed that the binding sites for p43, p60 and p120 were located primarily in two fragments (a and c). The last fragment (e) showed weaker and variable interactions with these proteins. Deletion of the a and c regions from the DMPK 3 ′UTR riboprobe resulted in significant loss of p43 and p60 interactions (last two lanes).

FIG. 3. Identification of DMPK 3 ′UTR RNA-binding proteins. HeLa nuclear protein extracts UV-crosslinked with a DMPK 3′UTR riboprobe (lane 1). UV-crosslinked RNA-protein complexes were digested with RNAse and then immunoprecipitated as indicated, lanes 2-5. The p60 complex consists of PTB and U2AF (lanes 3, 4), p43 is hnRNP C (lane 2) and PSF is a component of the p120 complex (lane 5). Interestingly, U2AF consistently co-precipitated with PSF (lane 5).

FIG. 4. DNA sequence of the 3′ end of the DMPK gene. Previously defined coding regions and their translation are in small letters. The DMPK 3′UTR is depicted in capital letters. 3′UTR sequences 5′ of the CTG repeats (upstream or up in the text) are in italics. The (CTG)ₙ tract is underlined. 3′UTR sequences 3′ of the repeats (downstream or dwn) are in bold. Exons are defined as E14, E15 and E16 (SEQ ID NO:1 which encodes SEQ ID NO:2, a 42 amino acid sequence) with exon junctions delineated by vertical lines. Relevant restriction enzyme recognition sites are indicated. Translation of the novel exon (E16) appears in bold capital letters below the DNA sequence. The potential PKC phosphorylation site is denoted by an (*). Boxes indicate sequences conforming to a consensus 3′ branch site (1), a polypyrimidine tract (2), a 3′ splice acceptor site (3) and a polyadenylation signal (4).

FIG. 5. The (CUG)ₙ tract is essential for E16+ splicing. RT-PCR results from transfections of C2C12 with a 3′ exon trapping vector (pTAG) encoding two adenovirus exons (A1, A2) separated by an intron. DMPK 3′UTR fragments with up to 100 CTGs were cloned 3′ of A2. The top band represents unspliced mRNA; the second band results from splicing of the adenovirus intron and the third band (bottom of gel) results from removal of all introns and E16 usage. CTG tracts of 5 to 100 repeats do not visibly affect E16 splicing. However, disruption of the CTG tract (last lane) results in complete suppression of splicing in to E16.

FIG. 6. Effect of CUG repeats on splicing. (A) RT-PCR-RFLP assay for quantifying the effects of CUG repeats. The gel depicts typical results of RT-PCR-RFLP assays on RNA extracted from C2C12 cells transfected with equal amounts of a control (C) plasmid [pTAG+DMPK 3′UTR (CTG)₅] and a tester (T) plasmid [pTAG+DMPK 3′UTR (CTG)_{5 or 57}]. The control plasmid (C) has been modified to eliminate a BamHI site in the DMPK 3′UTR. Lanes marked (U) contain fully spliced RT-PCR products (arrow) and partially spliced products (weaker bands at the top of the gel) from both plasmids. The completely spliced product has two exons from pTAG (A1 and A2) spliced to E16 from DMPK. PCR products are 3′end-labeled with ³²P (*). Digestion with BamHI (D) results in diminution of the signal (arrow) and the generation of a smaller end-labeled fragment (bottom of gel) corresponding to RT-PCR products derived from the tester plasmid. (B) Quantitation of the effect of the DM mutation on splice site usage. Using the RT-PCR RFLP assay, the amount of E16+ mRNA from the tester (T) and control (C) plasmids (arrow in FIG. 4A) was quantified as indicated. Five to six independent measurements were made for each tester (p<0.025, Wilcoxon rank sum test). Error bars represent 2×SEM. The presence of a (CTG)ₙ expansion from 57-100 has a deleterious effect on splicing, resulting in E16+ mRNA levels of approximately 30-35% as compared to mRNA levels from the wild type allele.

FIG. 7. DM mutation causes imbalance in relative levels of cytoplasmic DMPK mRNA isoforms. (A) Tissue distribution of E16+ mRNA. RT-PCR of DMPK mRNA isoforms using total RNA from various tissues of a 5 month old male infant (1=E16−PCR control, 2=E16+PCR control, 3=abdominal muscle, 4=diaphragm, 5=heart, 6=testes, 7=psoas muscle, 8=lung, 9=kidney, and 10=liver). Top two bands (E16−) are from CUG containing mRNAs. The bottom band is from the novel isoform (E16+) and constitutes about 10-15% of total DMPK mRNA is muscle tissues. (B-C) RT-PCR results from DM fibroblasts show that CUG containing mRNAs (E16−) from the mutant (mut) allele are completely trapped in the nucleus (N), while wild type (wt) mRNAs are effectively transported to the cytoplasm (C). Number of CTGs for each cell line is indicated. RT-PCR results for the novel mRNA isoform (E16+) show that transcripts from the mutant allele are effectively transported to the cytoplasm.

### DETAILED DESCRIPTION OF THE INVENTION

Definitions

As used herein, the terms “isolated and/or purified” refer to in vitro preparation, isolation and/or purification of a nucleic acid molecule or polypeptide of the invention from its natural cellular environment, and from association with other components of the cell, such as nucleic acid or polypeptide, so that it can be sequenced, replicated, and/or expressed. For example, “isolated DMPK nucleic acid” is RNA or DNA containing greater than 9, preferably 36, and more preferably 45 or more, sequential nucleotide bases that encode at least a portion of DMPK, e.g., DMPK RNA comprising at least a portion of exon 16, or a RNA or DNA complementary thereto, that is complementary or hybridizes, respectively, to RNA or DNA encoding DMPK or a particular isoform thereof, and remains stably bound under stringent conditions, as defined by methods well known in the art, e.g., in Sambrook et al. (Molecular Cloning: A Laboratory Manual, Cold Spring Harbor Laboratory, NY (1989)). Thus, the RNA or DNA is “isolated” in that it is free from at least one contaminating nucleic acid with which it is normally associated in the natural source of the RNA or DNA and is preferably substantially free of any other mammalian RNA or DNA. The phrase “free from at least one contaminating source nucleic acid with which it is normally associated” includes the case where the nucleic acid is reintroduced into the source or natural cell but is in a different chromosomal location or is otherwise flanked by nucleic acid sequences not normally found in the source cell. An example of isolated nucleic acid within the scope of the invention is RNA or DNA that encodes human DMPK comprising SEQ ID NO:2, e.g., which is encoded by DNA comprising SEQ ID NO:1.

As used herein, the term “recombinant nucleic acid” or “preselected nucleic acid,” e.g., “recombinant DNA sequence or segment” or “preselected DNA sequence or segment” refers to a nucleic acid, e.g., to DNA, that has been derived or isolated from any appropriate tissue source, that may be subsequently chemically altered in vitro, so that its sequence is not naturally occurring, or corresponds to naturally occurring sequences that are not positioned as they would be positioned in a genome which has not been transformed with exogenous DNA. An example of preselected DNA “derived” from a source, would be a DNA sequence that is identified as a useful fragment within a given organism, and which is then chemically synthesized in essentially pure form. An example of such DNA “isolated” from a source would be a useful DNA sequence that is excised or removed from said source by chemical means, e.g., by the use of restriction endonucleases, so that it can be further manipulated, e.g., amplified, for use in the invention, by the methodology of genetic engineering.

Thus, recovery or isolation of a given fragment of DNA from a restriction digest can employ separation of the digest on polyacrylamide or agarose gel by electrophoresis, identification of the fragment of interest by comparison of its mobility versus that of marker DNA fragments of known molecular weight, removal of the gel section containing the desired fragment, and separation of the gel from DNA. Therefore, “preselected DNA” includes completely synthetic DNA sequences, semi-synthetic DNA sequences, DNA sequences isolated from biological sources, and DNA sequences derived from RNA, as well as mixtures thereof.

As used herein, the term “carrier” includes KLH, tetanus toxoid, diphtheria toxoid ovalbumin, leukotoxin polypeptides, Pseudomonas exotoxin and sperm whale myoglobin, which produce an immune response.

As used herein, the term “derived” with respect to a RNA molecule means that the RNA molecule has complementary sequence identity to a particular DNA molecule.

A “recombinant” animal, e.g., a mouse, of the invention has a genome that has been manipulated in vitro so as to alter, e.g., decrease or disrupt, or, alternatively, increase, the function or activity of at least one isoform of DMPK.

Antibodies of the Invention

The antibodies of the invention are prepared by using standard techniques. To prepare polyclonal antibodies or “antisera,” an animal is inoculated with an antigen, i.e., a purified immunogenic DMPK peptide or polypeptide, and immunoglobulins are recovered from a fluid, such as blood serum, that contains the immunoglobulins, after the animal has had an immune response. For inoculation, the antigen is preferably a fusion peptide, e.g., a DMPK peptide linked to a carrier peptide or other heterologous peptide, e.g., a glutathione S transferase (GST) peptide, and preferably emulsified using a biologically suitable emulsifying agent, such as Freund's incomplete adjuvant. A variety of mammalian or avian host organisms may be used to prepare polyclonal antibodies against DMPK, e.g., goat, rabbit, mouse, rat, donkey, chicken and the like. Following immunization, Ig is purified from the immunized bird or mammal. For certain applications, particularly certain pharmaceutical applications, it is preferable to obtain a composition in which the antibodies are essentially free of antibodies that do not react with the immunogen. This composition is composed virtually entirely of the high titer, monospecific, purified polyclonal antibodies to DMPK, or certain isoforms thereof. Antibodies can be purified by affinity chromatography, using purified DMPK, a peptide thereof, or a ligand of the heterologous peptide. Purification of antibodies by affinity chromatography is generally known to those skilled in the art (see, for example, U.S. Pat. No. 4,533,630). Briefly, the purified antibody is contacted with purified DMPK or a peptide thereof, or the ligand of the heterologous peptide, bound to a solid support for a sufficient time and under appropriate conditions for the antibody to bind to the polypeptide, peptide or ligand. Such time and conditions are readily determinable by those skilled in the art. The unbound, unreacted antibody is then removed, such as by washing. The bound antibody is then recovered from the column by eluting the antibodies, so as to yield purified, monospecific polyclonal antibodies.

Monoclonal antibodies can be also prepared, using known hybridoma cell culture techniques. In general, this method involves preparing an antibody-producing fused cell line, e.g., of primary spleen cells fused with a compatible continuous line of myeloma cells, and growing the fused cells either in mass culture or in an animal species, such as a murine species, from which the myeloma cell line used was derived or is compatible. Such antibodies offer many advantages in comparison to those produced by inoculation of animals, as they are highly specific and sensitive and relatively “pure” immunochemically. Immunologically active fragments of the present antibodies are also within the scope of the present invention, e.g., the F(ab) fragment scFv antibodies, as are partially humanized monoclonal antibodies.

Thus, it will be understood by those skilled in the art that the hybridomas herein referred to may be subject to genetic mutation or other changes while still retaining the ability to produce monoclonal antibody of the same desired specificity. The present invention encompasses mutants, other derivatives and descendants of the hybridomas.

It will be further understood by those skilled in the art that a monoclonal antibody may be subjected to the techniques of recombinant DNA technology to produce other derivative antibodies, humanized or chimeric molecules or antibody fragments which retain the specificity of the original monoclonal antibody. Such techniques may involve combining DNA encoding the immunoglobulin variable region, or the complementarity determining regions (CDRs), of the monoclonal antibody with DNA coding the constant regions, or constant regions plus framework regions, of a different immunoglobulin, for example, to convert a mouse-derived monoclonal antibody into one having largely human immunoglobulin characteristics (see EP 184187A, 2188638A, herein incorporated by reference).

The antibodies of the invention are useful for detecting or determining the presence or amount of DMPK or certain isoforms thereof in a sample, e.g., a physiological sample such as a mammalian tissue biopsy or a mammalian physiological fluid, e.g., serum. The antibodies are contacted with the sample for a period of time and under conditions sufficient for antibodies to bind to the polypeptide so as to form a binary complex between at least a portion of said antibodies and said polypeptide. Such times, conditions and reaction media can be readily determined by persons skilled in the art.

For example, the physiological sample may be obtained from a mammal, e.g., a human. For samples comprising cells, the cells are lysed to yield an extract which comprises cellular proteins. Alternatively, intact cells, e.g., a tissue sample such as paraffin embedded and/or frozen sections of biopsies, are permeabilized in a manner which permits macromolecules, i.e., antibodies, to enter the cell. The antibodies of the invention are then incubated with the protein extract, e.g., in a Western blot, or permeabilized cells, e.g., prior to flow cytometry, so as to form a complex. For physiological fluid samples, the antibodies are contacted with the sample so as to form a complex. The presence or amount of the complex is then determined or detected.

The antibodies of the invention may also be coupled to an insoluble or soluble substrate. Soluble substrates include proteins such as bovine serum albumin. Preferably, the antibodies are bound to an insoluble substrate, i.e., a solid support. The antibodies are bound to the support in an amount and manner that allows the antibodies to bind the polypeptide (ligand). The amount of the antibodies used relative to a given substrate depends upon the particular antibody being used, the particular substrate, and the binding efficiency of the antibody to the ligand. The antibodies may be bound to the substrate in any suitable manner. Covalent, noncovalent, or ionic binding may be used. Covalent bonding can be accomplished by attaching the antibodies to reactive groups on the substrate directly or through a linking moiety.

The solid support may be any insoluble material to which the antibodies can be bound and which may be conveniently used in an assay of the invention. Such solid supports include permeable and semipermeable membranes, glass beads, plastic beads, latex beads, plastic microtiter wells or tubes, agarose or dextran particles, sepharose, and diatomaceous earth. Alternatively, the antibodies may be bound to any porous or liquid permeable material, such as a fibrous (paper, felt etc.) strip or sheet, or a screen or net. A binder may be used as long as it does not interfere with the ability of the antibodies to bind the ligands.

The invention also comprises reagents and kits for detecting the presence or amount of a particular isoform of DMPK or the relative amount of DMPK isoforms. Preferably, the reagent or kit comprises the purified antibodies of the invention in a liquid that does not adversely affect the activity of the antibodies in the intended assay. Preferably, the liquid is saline solution. Alternatively, the reagent or kit may comprise the purified antibodies attached to a substrate as discussed above. Preferably, the substrate is an insoluble solid support, e.g., the well of a microtiter plate. An alternative preferred substrate is solid particles, most preferably latex beads.

The diagnostic kit comprises, in a container or packaging, one or more of the reagents of the invention and a means for detecting or measuring the formation of complexes created by the binding of polypeptide and the antibodies in the reagents. The detecting or measuring means is preferably an immunoassay, such radioimmunoassay, enzyme-linked immunosorbent assay (ELISA), or an immunofluorescence assay. Most preferably, the detecting or measuring means is a reagent capable of binding to the complexes formed by DMPK and the antibodies and containing a detectable moiety. Such reagent may be the antibody of the invention conjugated with a detectable moiety. Alternatively, the antibody can be a second antibody, which is an antibody which binds to the antibodies of the invention, conjugated to a detectable moiety.

The invention will be further described by the following non-limiting example.

### EXAMPLE

Experimental Procedures

Nuclear and cytoplasmic protein extracts

Cells were resuspended at 10⁷-10⁸ cells/ml in CEB (10 mM Tris-HCl pH 7.6, 1.5 mM MgCl₂, 10 mM KCl, 0.5 mM EDTA, and 1 mM DTT) with protease inhibitors and incubated on ice for 20 minutes. Triton X-100 (0.5% v/v) was added and cells were disrupted by 40 strokes through a G25 hypodermic needle. Nuclei were centrifuged for 15 minutes at 2000 g. The supernatant was saved (cytoplasmic extract). The nuclear pellet was resuspended in NEB (20 mM Tris-HCl pH 7.6, 25% sucrose, 420 mM NaCl, 1.5 mM MgCl₂, and 0.5 mM DTT) plus protease inhibitors and incubated for 40 minutes on ice, then centrifuged for 10 minutes at 10,000 g. The supernatant (nuclear extract) was dialyzed against 20 mM Tris-HCl pH 7.6, 20% glycerol, 20 mM KCl, 1.5 mM MgCl₂, 0.2 mM EDTA, and 1 mM DTT. Extracts were flash frozen and stored at −80° C. until use.

UV Crosslink assays Riboprobes were labeled to high specific activity using ³²P-UTP and the Riboprobe (Promega) kit according to the manufacturer's protocol. Protein extracts (20 μg) were mixed with the riboprobe (8 fmol) in 15 mM Hepes, pH 7.9, 50 mM KCl, 10% glycerol, 0.2 mM DTT, and 0.2 mg/ml tRNA in a final volume of 10 μl. Excess competitor RNA was transcribed using Maxiscript (Ambion) as per manufacturer's protocol and added if required. After a 20 minute incubation at 25° C., the reactions were put on ice and irradiated with UV light (Stratalinker 2400, Stratagene) for 10 minutes. Samples were digested with 24 μ RNAse T1 and 10 μg RNAse A for 45 minutes at 37° C. and resolved by 10% SDS-PAGE.

Immunoprecipitations

Twenty reactions (as above) were UV-crosslinked, digested with RNAse and then diluted to 500 μl in NET buffer (150 mM NaCl, 50 mM Tris-HCl pH 7.4, and 1 mM EDTA). Antibodies were added and the samples were incubated for 1 hour at 4° C. Sepharose-protein A/G beads (Pharmacia) (30 μl) were added and the incubation continued for 1 hour. The beads were pelleted and washed 5 times in NET buffer containing 0.5% deoxycholic acid, 0.1% SDS and 0.5% NP40. Immunoprecipitated complexes were resolved by SDS-PAGE.

Anti-hnRNP C, anti-hnRNP L and anti-hnRNP U were provided by G. Dreyfuss, anti-CUG-BP by L. Timchenko and M. Swanson, anti-PSF by J. G. Patton, anti-nucleolin by J. Malter, anti-PTB by D. Heifman and S. Huang, and anti-U2AF by M. Carmo-Fonseca.

Cell culture and transfections

DM fibroblasts were grown as described (Hamshere et al., 1997). C2C12 mouse myoblasts were grown in Dulbecco's modified Eagle's medium (DMEM; Cellgro, Herndon, Va.)+10% cosmic calf serum (HyClone, Logan, Utah) and transfected with 2 μg of plasmid constructs per 7×10⁵ cells using Lipofectamine PLUS reagent (Gibco BRL) according to manufacturer's protocol. Cells were grown for 24-48 hours and then harvested.

RNA extracts

For total RNA extracts, cells were resuspended in thiocyanate buffer (4 M guanidinium thiocyanate, 20 mM NaOAc pH 5.4, and 0.5% sarkosyl) and incubated for 15 minutes on ice. The homogenate was extracted twice with phenol-chloroform (5:1, pH 3.5), ethanol precipitated and resuspended in H₂O. When required, RNA preparations were subject to RNAse free DNAse digestion, re-extracted with acid phenol-chloroform and reprecipitated before RT-PCR. Tissues were ground for 30 seconds in ice-cold suspension buffer (100 mM NaCl, 10 mM Tris-HCl pH 7.6, and 1 mM EDTA, plus protease inhibitors) using a hand held tissue homogenizer and clarified by centrifugation. Subsequently, thiocyanate buffer was added and samples treated as above. For nuclear/cytoplasmic fractionations, cells were trypsinized and resuspended in buffer (10 mM Tris-HCl, pH 8, 0.14 M NaCl, 1.5 mM MgCl₂) for 30 minutes on ice. NP40 was added to 0.65% final concentration and the cells were disrupted by 20 strokes through a G23 hypodermic needle. Nuclei were pelleted for 15 minutes at 2500 g, washed in hypotonic buffer, repelleted and resuspended in thiocyanate buffer (nuclear fraction). Both supernatants (cytoplasmic fraction) were pooled and diluted 1:1 in thiocyanate buffer. RNA from both fractions was pelleted by ultracentrifugation for 15 hours at 18° C., 30,000 rpm through a 5.7 M CsCl cushion. RNA pellets were ethanol washed and resuspended in H₂O.

RT-PCR

RNA (2-10 μg) was denatured for 10 minutes at 70° C. and mixed with RT buffer (Promega), primer and 5 units of MLTV-RT (Promega) in a 20 μl final volume. RT primers were #212 (see Table 1 below) for detection of the E16−-isoforms, and #57 (Table 1) for all other assays. Reactions were incubated for 1 hour at 37° C. Two to five μl of RT reaction were used for PCR amplification. For quantitative measurements, the 3′ primer was end labeled with (γ-³²P ATP) using T4 polynucleotide kinase. Both E16− and E16+ isoforms were amplified using primers #9003 (Table 1) and #11111 (Table 1) and 10 cycles of 95° C. for 60 seconds, 60° C. for 60 seconds and 72° C. for 90 seconds, followed by 30 cycles of 95° C. for 60 seconds, 55° C. for 60 seconds and 72° C. for 90 seconds. PCR products were run on 1.5% agarose gels and the signal was quantified by phosphorimaging (Molecular Dynamics). cDNAs derived from pTAG constructs were PCR amplified using primers #148 (Table 1) and #11 (Table 1) and 25 cycles of 95° C. for 60 seconds, 60° C. for 60 seconds and 72° C. for 90 seconds. PCR products were digested with the appropriate restriction enzyme if required by the experiment and quantified as above.

For PCR detection of the E16− isoforms in DM fibroblast cell lines, primers #206 (end labeled) (Table 1) and #27 (Table 1) were used in 30 cycles of 95° C. for 60 seconds, 60° C. for 60 seconds and 72° C. for 90 seconds. Detection of the E16+ isoform in DM fibroblasts required two rounds of PCR amplification due to its low abundance. For the first round, primers #205 (Table 1) and #200 (Table 1) were used in 40 cycles of 95° C. for 60 seconds, 60° C. for 60 seconds and 72° C. for 90 seconds. Two μl of the resulting PCR product were reamplified by nested PCR using primers #206 (end labeled) (Table 1) and #200 (Table 1). PCR products were digested with BpmI (NEB), run on 1% agarose, 2% NuSieve gels, and quantified as above.

```
TABLE 1
  Primer 212: 5′-ACTGGAGCTGGGCGGAGACCCA-3′ (−127 to −106
from E15 stop codon)(SEQ ID NO:3)
  Primer 57: 5′-GGGCAGATGGAGGGCCTT-3′ (17 bases 3′ of
DMPK pA signal)(SEQ ID NO:4)
  Primer 9003: 5′-GCTGAAGTGGCAGTTCCA-3′ (5′ −3′
in DMPK E11)(SEQ ID NO:5)
  Primer 11111: 5′-TGTCGGGGTCTCAGTGCATCCA-3′ (55 bases 5′
of DMPK pA signal)(SEQ ID NO:6)
  Primer 148: 5′-CTCTTCGCATCGCTGTCTGCGAG-3′ (in A1
of pTAG)(SEQ ID NO:7)
  Primer 11: 5′-GTCGGGGGTGGGGGTCCAT-3′ (7 bases 5′
of DMPK pA signal) (SEQ ID NO:8)
  Primer 206: 5′-GGGGTCCACCTGCCTTTTGT-3′ (5′ −3′
in DMPK E9)(SEQ ID NO:9)
  Primer 27: 5′-GAGTCGAAGACAGTTCTAG-3′ (−21 to −3
from DMPK E15 stop codon)(SEQ ID NO:10)
  Primer 205: 5′-GGGAGACACTGTCGGACATT-3′ (5′ −3′
in DMPK E9)(SEQ ID NO:11)
  Primer 200: 5′-ACGTCAGGGCCTCAGCCCT-3′ (spans DMPK
E16-E14 splice junction)(SEQ ID NO:12)
```

RNAse Protection Assay

In order to quantify the effect of the DM mutation on DMPK mRNA isoform ratios, in addition to the RT-PCR assays, a method was developed based on the RNAse protection assay. The ribonuclease protection assay is an extremely sensitive procedure for the detection and quantitation of RNA species in a complex sample mixture of total or polyA RNA. For the RPA, a labeled (nonisotopic or radioactive) RNA probe is made that is complementary to part of the target RNA to be analyzed. This is done by placing the 3′ end of the probe sequence adjacent to one of the phage polymerase promoters (T7, T3 or SP6) by cloning into a plasmid vector or by using a PCR primer that contains the promoter sequence. The corresponding RNA polymerase is then used to generate an antisense RNA transcript by in vitro transcription. The labeled probe and sample RNA are incubated under conditions that favor hybridization of complementary sequences. After hybridization, the mixture is treated with RNAse to degrade unhybridized RNA. Labeled probe that is hybridized to complementary RNA from the sample is protected from RNAse digestion and can be separated by polyacrylamide gel electrophoresis and visualized by autoradiography or by a secondary detection method. When the probe is present in molar excess over the target in the hybridization reaction, the intensity of the protected fragment will be directly proportional to the amount of target RNA in the sample mixture.

Thus probes spanning any part of the DMPK E16 and the intron that precedes the exon, can be used to detect E16+ and E16− DMPK isoforms. Similarly, probes that span E16 and another exon such as E14 for example, can be used to detect and quantitate that specific RNA in a complex mixture. This technique was used to study the various DMPK isoforms in RNAs extracted from DM or non-DM cells (eg. fibroblasts, myoblasts) or tissues (eg. muscle biopsies).

The following sequences are examples of antisense RNA that can be used in the RPA assay. They read from the 5′ to 3′end of the probe.

| | | |
|---|---|---|
| 1) | cccacuuugcgaaccaacgauaggugggggugcguggaggauggaacacggacggcccggcu | (SEQ ID NO:16) |
| | ugcugccuucccaggccugcaguuugcccauccacgucagggccucagcCUGGCCGAA | |
| | AGAAAGAAAUGGUCUGUGAUCCCCCCGCUAUAGGCUGAACCACGAU | |
| 2) | gaggaggggggcgcgggauccccgaaaaaagcggguuuggcaaaagcaaauuucccgaguaag | (SEQ ID NO:17) |
| | caggcagagaucgcgccacacgcuccccagagcagggcgucaugcacaagaaagcuuugcacu | |
| | uugcgaaccaacgauaggugggggugcgcggaggauggaacacggacggcccggcuugcugc | |
| | cuucccaggccugcaguuugcccauccacgucagggccucagcCUGGCCGAAAGAAA | |
| | GAAAUGGUCUGUGAUCCCCCCGCUAUAGGCUGAACCACGAU | |
| 3) | gaggaggggggcgcgggauccccgaaaaagcggguuuggcaaaagcaaauuucccgaguaag | (SEQ ID NO:18) |
| | caggcagagaucgcgccacacgcuccccagagcagggcgucaugcacaagaaagcuuugcacu | |
| | uugcgaaccaacgauaggugggggugcgcggaggauggaacacggacggcccggcuugcugc | |
| | cuucccaggccugcaguuugcccauccacgucagggccucagcccuggcagggagcagcaggug | |
| | gcggcggugcauggggccuggccccaccagAUAUAGUCAACGUCGCGUAC | |

For all three probes, the bold letters represent antisense sequences to E16 or the 3′UTR that follows it. The capital letters represent antisense sequences to the intron that precedes E16 up to the CUG repeats. The italicized capital letters represent a tag sequence that is used to distinguish undigested riboprobe from digested probe. The non-bold small letters in probe number 3 represent the antisense sequences to E14 of DMPK.

Results

Detection of specific RNA-binding proteins interacting with the DMPK 3′UTR

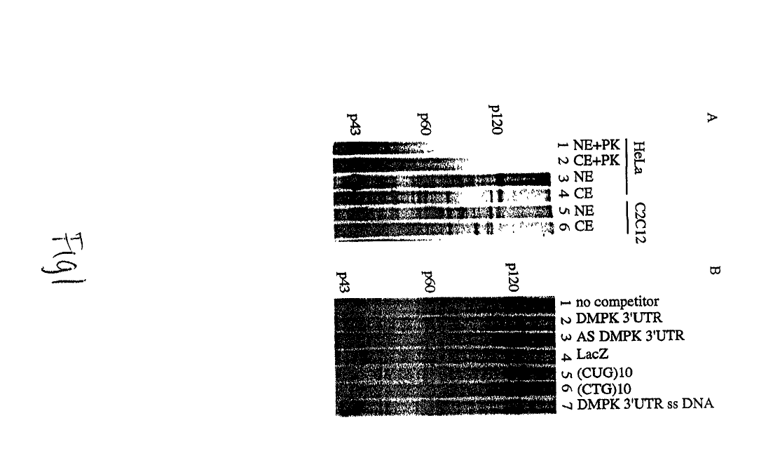

It has been proposed that the CUG tract could constitute a binding site for CUG binding proteins and that the interaction could be affected by the DM mutation (Caskey et al., 1996; Timchenko et al., 1996). Several groups have identified proteins capable of binding CUG repeats using CUG oligonucleotide riboprobes in gel shift or UV-crosslinking assays (Bhagwati et al., 1996; Timchenko et al., 1996). Following a broader approach, the whole DMPK 3′UTR was employed as a probe, as the CUG expansion could affect the interaction between the 3′UTR and RNA-binding proteins without necessarily being a binding site itself. A UV-crosslinking assay using a full length DMPK 3′UTR riboprobe containing 5 CUG repeats revealed a number of distinct RNA-protein complexes in nuclear and cytoplasmic protein extracts from HeLa cells (FIG. 1A). The absence of complexes using protein extracts treated with proteinase K (FIG. 1A) and the presence of complexes despite competition with great excesses of DNA probes (FIG. 1B) verified that these were RNA-protein complexes. Three prominent groups of bands were consistently present in the nuclear extract and are referred to by their approximate molecular weight as p43 (consisting of a doublet), p60 (formed by three distinct bands: p60, p62 and p65) and p120. Other bands of 50 and 75 kDa were also detected. The overall pattern was similar in the cytoplasmic fraction, although relative abundance differed: p43, p60, p75 and p120 were predominantly nuclear, while p50 was more abundant in the cytoplasm. Similar results were seen using C2C12 mouse myoblast protein extracts (FIG. 1A) and human fibroblasts (data not shown). No major differences were noted with a riboprobe consisting of the DMPK 3′UTR with 130 CUG repeats (data not shown).

Competition assays with 400×molar excess of unlabeled DMPK 3′UTR RNA resulted in almost complete loss of signal, whereas addition of the same amount of unlabeled antisense DMPK 3′UTR RNA had no significant effect (FIG. 1B). A second heterologous competitor, derived from the E. coli β-galactosidase gene, showed a more complex behavior. It effectively competed for p120 and p43, but had no effect on p60 (FIG. 1B), suggesting different binding specificity of the proteins involved. Competition with a (CUG)₁₀ oligonucleotide did not affect p120, p43 and p65, but did compete for p60 and p62, although not as effectively as DMPK 3′UTR RNA itself (FIG. 1B). Thus, the complexes detected by this UV-crosslinking assay involved RNA-binding proteins exhibiting a certain degree of specificity.

RNA-binding proteins interact with two distinct sequences 3′ to CUG repeats

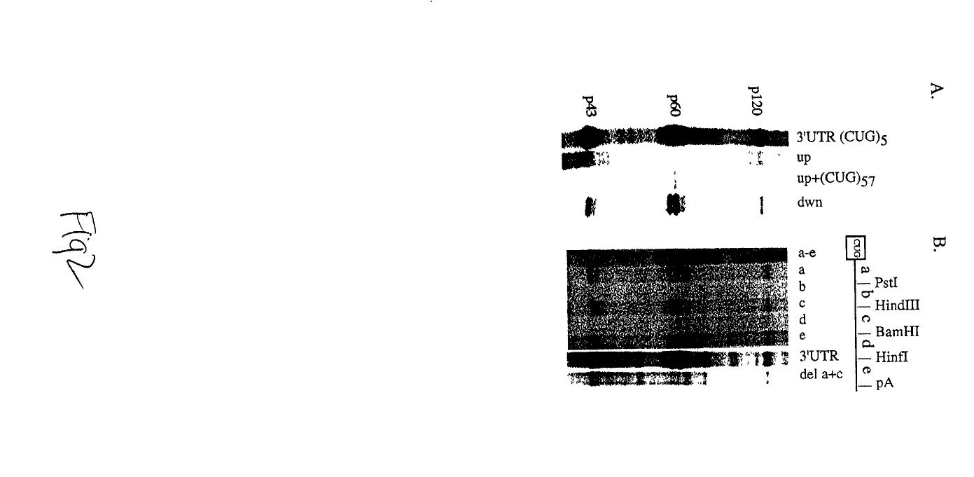

To determine which regions of the DMPK 3′UTR RNA were responsible for these interactions, the 3′UTR was divided into three sections: 1) 5′ of the CUG repeats (upstream or up), 2) CUG repeats, and 3) 3′ to the CUG repeats (downstream or dwn). Riboprobes consisting of up, up+(CUG)₅₇ and dwn revealed that the sequences primarily responsible for binding p43, p60 and p120 were clearly located 3′ to the CUG repeats (FIG. 2A). To further delineate the binding sites, riboprobes corresponding to 5 non-overlapping sections spanning the downstream region were employed (FIG. 2B). Two sequences of about 70 (a, FIG. 2B) and 100 (c, FIG. 2B) nucleotides were consistently capable of interaction with p43, p60 and p120. To confirm these results, a riboprobe was tested that contained the complete 3′UTR in which the putative binding sites had been deleted (FIG. 2B). Interaction of the RNA-binding proteins with this mRNA was affected to different degrees. Interactions with p43 and p60 (p60 and p62 in particular) were severely diminished. In contrast, interaction with p120 was much less affected. Thus, these two regions (a and c, FIG. 2B) contain the sequences primarily responsible for binding to p43 and particularly p60.

Identification of DMPK 3′UTR RNA-binding proteins

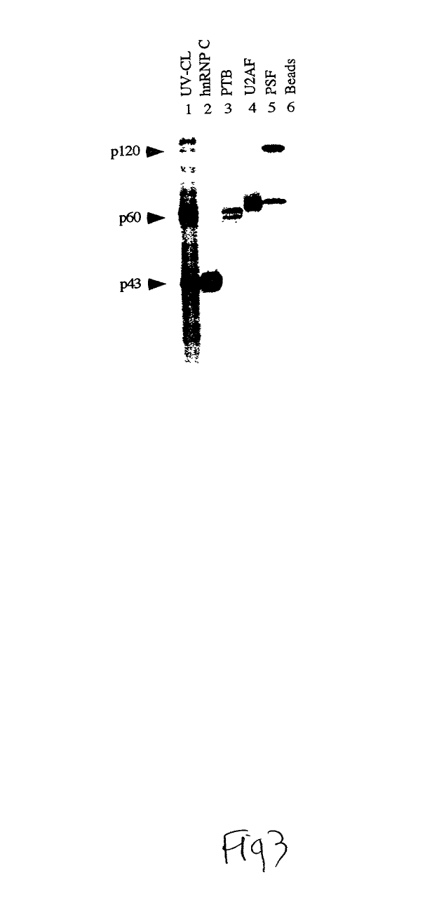

Suspecting that the proteins detected by the UV-crosslinking assay might be previously characterized RNA-binding proteins such as hnRNP's or splicing factors, the UV-crosslinked complexes were immunoprecipitated. Four antisera were consistently capable of specifically immunoprecipitating different complexes (FIG. 3). The p43 doublet was recognized by anti-hnRNP C. Of the p60 group, p60 and p62 were immunoprecipitated by anti-PTB, while p65 was immunoprecipitated by anti-U2AF. Finally, the lower band of the p120 group was recognized by anti-PSF. In all four cases, the sizes of the UV-crosslinked complexes were consistent with the sizes of hnRNP C, PTB, U2AF and PSF (Choi and Dreyfuss, 1984; Garcia-Blanco et al., 1989; Patton et al., 1993; Zamore and Green, 1989). Furthermore, hnRNP C from human cells has been reported to run as a doublet by SDS-PAGE (Choi and Dreyfuss, 1984) and as a triplet in protein extracts from mouse tissues (Kamma et al., 1995), as is the case for the p43 group (see FIG. 1A). Similarly, PTB has been reported to run as a doublet (p60 and p62 in the UV-crosslink assay described herein) (Garcia-Blanco et al., 1989). Thus, the immunoprecipitation results strongly suggest that hnRNP C, PTB, U2AF and PSF bind to the DMPK 3′UTR mRNA. Interestingly, immunoprecipitation of UV-crosslinked complexes with anti-PSF resulted in the co-precipitation of U2AF in addition to PSF (FIG. 3). The converse was also seen when anti-U2AF was used for immunoprecipitation, though not as consistently. Sera against several other RNA-binding proteins (hnRNP L, hnRNP U, nucleolin and CUG-BP) were also tested and proved negative (data not shown).

Evidence for a novel DMPK exon located 3′ to the CUG repeats

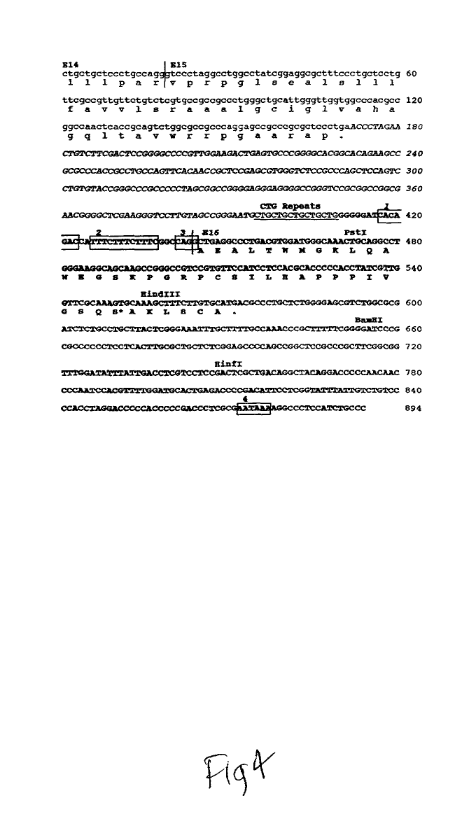

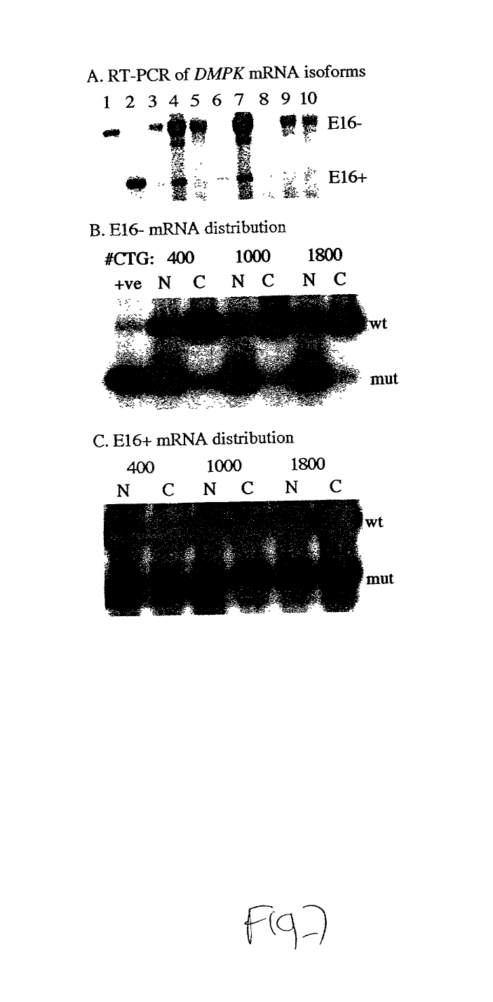

All four RNA-binding proteins that were identified (PTB, U2AF, PSF and hnRNP C) have been shown to have a role or been implicated in the control of RNA splicing (Choi et al., 1986; Lou et al., 1999; Patton et al., 1993; Zamore and Green, 1989). Furthermore, each of them has been shown to bind to intron polypyrimidine tracts or the 3′ end of introns (Garcia-Blanco et al., 1989; Patton et al., 1993; Swanson and Dreyfuss, 1988; Zamore and Green, 1989). Careful examination of one of the binding regions within the DMPK 3′UTR revealed a potential splice acceptor site. A consensus branch site, a polypyrimidine tract and a consensus 3′ splice site are all located within 40 nucleotides 3′ to the CUG repeats (FIG. 4). Another short polypyrimidine tract is found in the second binding region between the HindIII and BamHI sites; however, no branch site or 3′ splice site is apparent. A cDNA clone in the dbEST database (accession number AA195148) has exon 14 (E14) of DMPK spliced into the novel 3′ splice site rather than into exon 15. This eliminates a sequence of 425 bases (including the CUG repeats) and incorporates a novel exon (termed E16) encoding a carboxy terminus of 42 amino acids. This new mRNA isoform (termed E16+) was detected by RT-PCR in several human tissues from an unaffected five-month old male infant (FIG. 7A) and its identity confirmed by DNA sequencing. E16+ shows tissue specificity, being most abundant in muscle and testis, and low or absent in heart, lung, liver and kidney. By RT-PCR using radiolabeled primers, the relative levels of E16+ in various skeletal muscles are estimated at 10-15% of total DMPK mRNA, similar to those of the second most common DMPK mRNA isoform. The novel isoform was also detected in uterus, psoas muscle and cerebellum of a normal adult female, but only after a second round of PCR using nested primers. In contrast, mRNA isoforms containing the CUG repeats (termed E16−) were detected easily with the first PCR amplification (data not shown). E16+ mRNA was absent by RT-PCR in non-DM muscle biopsies from three other women and one man (data not shown). This suggests that this splicing event is developmentally regulated.

The role of CUG repeats in splicing

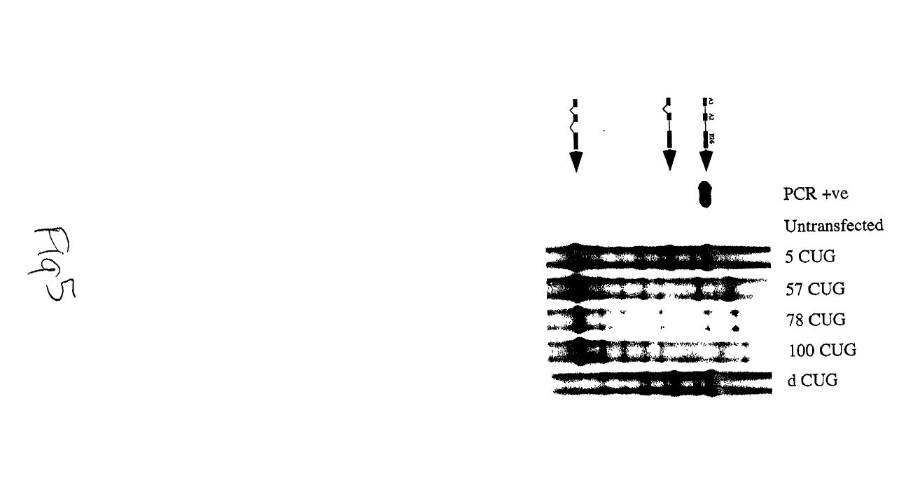

To determine the effect of CTG length on usage of the novel 3′ splice site, normal and mutated DMPK 3′UTR fragments were cloned into a 3′ exon trapping vector (pTAG, Gibco BRL). This construct consists of a CMV promoter and two short adenovirus exons separated by an intron. The DMPK 3′UTR was cloned immediately after adenovirus A2. Transient transfection of constructs into C2C12 mouse myoblasts and subsequent RT-PCR assays on the extracted RNA were used to detect the various mRNA isoforms generated. When a DMPK 3′UTR with 5 CTG repeats was tested, three bands were detected (FIG. 5). The top two bands represented unspliced mRNA and a mRNA that had spliced out the adenovirus intron, respectively. The lowest band was shown by DNA sequencing to have undergone a splicing event that resulted in the joining of adenovirus E2 to E16 of DMPK. Expansions of up to 100 repeats did not visibly affect the capability of the splice site to function as a splice acceptor (FIG. 5). But, remarkably, disruption of the 5 CTG repeats (aatg[CTG]₅gggg to gatc[CTGCAGCTCTG]gggg) resulted in complete suppression of splicing into the 3′ splice site (FIG. 5). This result strongly suggests the CUG repeats of the DMPK mRNA are an essential cis acting element required for this splicing event, and clearly showed that an expansion of up to 100 CUG repeats did not block splicing.

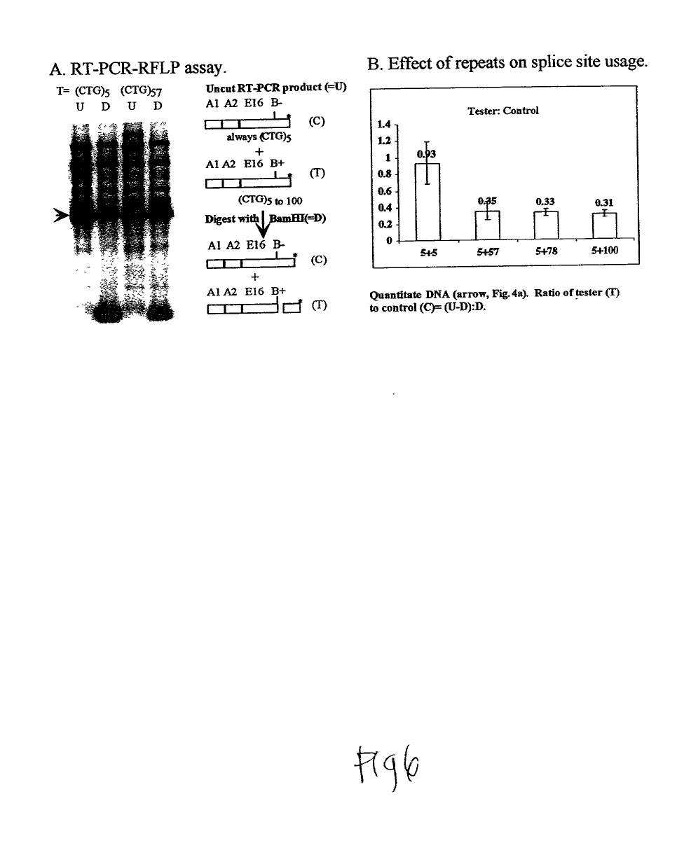

To quantify the effect of CTG expansions on the splice site usage more precisely, pTAG+DMPK 3′UTR (CTG)₅ was modified so as to disrupt a unique BamHI site located 243 bp 3′ to the repeats. This plasmid, lacking the BamHI site (B−) was used as the “control” allele. A quantitative PCR assay was established by testing mixtures of different ratios of pTAG+DMPK 3′UTR (CTG)₅ with (B+) and without (B−) the BamHI site. PCR parameters were established under which the ratio of products corresponding to each allele in the PCR reaction output reflected the ratio of the plasmid template input, indicating that the PCR assay was indeed capable of detecting concentration differences between both alleles. pTAG+DMPK 3′UTR (CTG)₅ (B−) (i.e., the “control” allele) was mixed in 1:1 ratio with pTAG DMPK 3′UTR (B+) constructs containing 5, 57, 78 or 100 CTG repeats (the “tester” allele). The different plasmid mixtures were co-transfected into C2C12 myoblasts, and quantitative PCR was done on DNA extracts from transfected cells to accurately determine the plasmid ratios present in each transfection. RT-PCR followed by comparison of undigested and BamHI digested samples was then used to analyze expression in an allele specific manner (FIG. 6A). Relative levels of each allele were normalized to DNA content, and a (B+)/(B−) ratio calculated for each co-transfection. Co-transfecting plasmid mixtures allowed us to look at ratios of the 3′ splice site usage from both alleles within the same cell, using the (CTG)₅ (B−) as an internal standard and eliminating the need for absolute quantitative RT-PCR. Thus, it could be determined if presence of increasing number of CTG repeats caused any departure from the input template ratio.

When plasmids (CTG)₅ (B−) and (CTG)₅ (B+) were co-transfected, the relative levels of spliced product incorporating E16 from the two alleles were not statistically different, as expected (FIG. 6B); the average (B+)/(B−) ratio (n=5) was 0.93. When the plasmids co-transfected had 5 CTG repeats versus 57, 78 or 100 CTG repeats, the average ratio was 0.35 (n=6), 0.33 (n=6) and 0.31 (n=5) respectively (FIG. 6B). These values differed significantly from the previous (B+)/(B−) ratio. Thus, a (CTG)₅₇₋₁₀₀ expansion has a deleterious effect on E16+ mRNA production, resulting in levels of approximately 30-35% of that from the normal (CTG)₅ allele.

To determine the effect of large CTG repeat tracts, three DM fibroblast cell lines (Alwazzan et al., 1999; Hamshere et al., 1997) (containing 400, 1000 and 1800 CTG repeats) heterozygous for a BpmI polymorphism in exon 10 were utilized. This polymorphism has been used previously to analyze allele specific expression (Hamshere et al., 1997; Krahe et al., 1995; Sabourin et al., 1993). When an allele specific assay was used to determine expression levels of the E16+ isoform in cytoplasmic total RNA extracts, the amount of E16+ mRNA from the mutant allele was approximately 50% (range of 46-65%) of that from the wild type allele (FIG. 7C). Thus, splicing into the novel splice site occurs at significant though reduced levels (as compared to mRNA from the wild type allele), even in the presence of large numbers of CTG repeats.

The DM mutation causes imbalance of relative levels of DMPK mRNA isoforms in the cytoplasm

The results just described show that even in presence of large numbers of repeats, a novel DMPK mRNA isoform lacking a CUG tract (and therefore presumably not subject to nuclear retention) is produced. It has been previously shown that increased numbers of CUG repeats in the mutant DMPK mRNA result in nuclear entrapment (Davis et al., 1997; Hamshere et al., 1997; Taneja et al., 1995). Thus, the DM mutation could cause an imbalance in relative levels of DMPK mRNA isoforms (E16− to E16+ ratio) in the cytoplasm. When the allele specific assay was used to determine expression levels of the E16− mRNA isoforms in nuclear and cytoplasmic total RNA extracts, the mRNA from the wild type allele was found mostly in the cytoplasmic fraction (FIG. 7B). In contrast, the mRNA from the DM allele was found exclusively in the nuclear fraction (FIG. 7B). This reproduced published data, and also indicated that nuclear leakage during cell fractionation was minimal. Remarkably, when the same RNA extracts were assayed for allele specific expression of the novel isoform, the E16+ mRNA from the mutant allele was clearly detected in the cytoplasm of all three DM cell lines tested (FIG. 7C). Thus, in vivo, while all of the E16− mRNA from the mutant allele is retained in the nucleus, a significant amount of E16+ mRNA is exported into the cytoplasm.

Discussion

The identification of the CTG triplet repeat expansion led to the hypothesis that CUG binding proteins could be titrated from nuclear pools by the DM mutation (Caskey et al., 1996). A number of CUG binding proteins have been detected (Bhagwati et al., 1996; Timchenko et al., 1996) and a 50 kD heterogeneous nuclear ribonucleoprotein, CUG-BP, intensely studied. A model has been proposed by which CUG expansions result in altered nucleo-cytoplasmic distribution of CUG-BP (Roberts et al., 1997). Enhanced nuclear levels of CUG-BP have been shown to affect regulation of alternative splicing of cardiac troponin T (cTNT), thus providing evidence for a role for CUG-BP in a trans effect mediated by the mutant DM transcript (Philips et al., 1998). However, there is no evidence for titration of CUG-BP by expanded CUG repeats, and the mechanism of intracellular CUG-BP redistribution remains unclear.

Reasoning that RNA-binding proteins could potentially interact with complex binding sites or binding sites other than the CUG tract, a search was undertaken for proteins capable of binding to portions of the DMPK 3′UTR mRNA which are not part of the CUG tract. If RNA-binding proteins interacted with complex binding sites or sites other than the CUG tract, a CUG expansion could affect those interactions without necessarily being itself a binding site. Furthermore, given that the nuclear foci detected in DM tissues contain full length DMPK mRNA (Taneja et al., 1995), the foci could act as sinks for interacting proteins, resulting in titration of such proteins from nuclear pools and a trans effect on other mRNAs. Using a combination of UV-crosslinking and immunoprecipitation, hnRNP C, PTB, U2AF and PSF were identified as proteins that interact specifically with sequences 3′ to the CUG repeats. The experiments described herein do not distinguish whether these proteins are binding as a complex or compete for overlapping binding sites. However, it was noted that although similar molar amounts of riboprobe were used, the intensity of the signal diminished notably as shorter probes were utilized. Thus, it is possible that the presence of other sequences in the 3′UTR strengthens or stabilizes the interaction with the two primary binding sites identified. Nonetheless, no difference in binding patterns or affinity of the RNA-protein complexes was observed when a DMPK 3′UTR+130 CUG repeats riboprobe was tested by UV-crosslinking.

Interestingly, immunoprecipitation of PSF resulted in coprecipitation of U2AF; the converse result was also observed, albeit not as consistently. This suggests a previously unreported interaction between U2AF and PSF. Unexpectedly, we were not able to detect CUG-BP despite repeated attempts. Efforts to colocalize the four RNA binding proteins we identified to RNA foci through combined immunohistochemistry and RNA-FISH were inconclusive due to the high nuclear signal observed (data not shown). Thus, even if foci do contain these RNA-binding proteins, the residual high nuclear protein levels rule out any significant titration effect.

Importantly, based on the identity and known functions of the RNA-binding proteins and subsequent sequence analysis, a novel intron-exon boundary downstream of the CUG repeats was identified. The new DMPK mRNA isoform splices DMPK E14 to the novel splice junction (and thus lacking 425 nucleotides, including the CUG tract). Moreover, this isoform was detected in vivo by RT-PCR. Expression was most abundant in skeletal muscle tissues and testis. The fact that the novel isoform was readily detected in tissues from a 5 month old individual but required two rounds of PCR to be detected in adult tissues clearly raises the possibility that expression of the E16+ isoform is developmentally regulated. Furthermore, PTB may have a role in this process, as PTB binding to a non-canonical PTB binding site of the DMPK 3′UTR results in repression of splicing at this splice site (unpublished data).

Remarkably, disruption of the normal (CUG)₅ resulted in complete inhibition of splicing. While CUG repeats have been implicated in regulation of alternative splicing of cTNT (Philips et al., 1998) and several neuron specific transcripts (Zhang et al., 1999), the results described herein provide the first evidence that the CUG repeats within the DMPK 3′UTR are an essential cis element for the utilization of E16. The CUG tract could function as a binding site for a trans acting regulator (CUG-BP or other) involved in regulation of alternative splicing of the novel exon. These results redefine the structure of the 3′ end of the DMPK gene; formally, the DM mutation lies in an intron and not the 3′UTR. Interestingly, the splice junction is absent in the mouse Dmpk 3′UTR and the CTG tract is imperfect (CTGCTGCAGCAGCTG; SEQ ID NO:13).

The effect of CUG expansions on E16+ DMPK mRNA isoform levels was also determined. Taken together, the in vitro and in vivo RT-PCR experiments show that expansions from 50 to greater than 1800 CTG repeats cause a reduction in E16+ mRNA levels from the DM allele to approximately 50% of levels from the wild type allele. Thus, DM cells likely suffer a decrease of total E16+ mRNA to levels of approximately 75% of levels found in normal cells.

Though a possible effect of the DM mutation on DMPK isoform ratio has been previously proposed (Jansen et al., 1992), it has received little attention. At least three mRNA isoforms of DMPK (full length, lacking E13 or lacking both E13 and E14), all containing CUG repeats in their 3′UTRs, have been reported (Jansen et al., 1992), but presence of the DM mutation resulted in no detectable difference on their relative levels (Krahe et al., 1995). The fact that usage of the novel splice site is not abolished even in the presence of large expansions led to the hypothesis that the DM mutation results in an alteration of the relative cytoplasmic levels of DMPK mRNA isoforms containing or lacking CUG repeats. The allele specific RT-PCR results on nuclear and cytoplasmic RNA fractions support this hypothesis. Consistent with previously published results (Davis et al., 1997; Hamshere et al., 1997), mRNA isoforms containing the CUG expansion suffer complete nuclear retention, while those with a normal number of CUG repeats are efficiently exported into the cytoplasm. The total cytoplasmic level of E16− DMPK mRNA in DM cells is therefore reduced to 50% of the level found in non-DM cells. In contrast, E16+mRNAs from both alleles are clearly detected in the cytoplasm. Even though the levels of the novel isoform (E16+) are reduced by the presence of CUG expansions, that amount which is produced is effectively exported to the cytoplasm. Therefore, the net effect of the DM mutation on the ratio of E16− to E16+ results in a relative overabundance of E16+ mRNA isoforms.

The E16+ isoform is not abundantly expressed in fibroblasts; indeed, its detection requires two rounds of PCR, whereas detection of the E16− isoforms requires only one. Thus, in fibroblasts the imbalance is minor. In tissues where expression of the novel isoform is comparable to that of other DMPK isoforms, such as in the skeletal muscle samples from the five-month old child, the imbalance could be significant, both in terms of the actual isoform ratios and functional consequences. Such an outcome has recently been described for Frasier syndrome, an autosomal dominant disorder in which a mutation in the exon 9 splice donor site of the WT1 gene results in an imbalance of WT1 isoforms in vivo (Barbaux et al., 1997; Klamt et al., 1998).

Thus, the CUG repeats play a role in regulation of alternative splicing of a novel DMPK isoform and evidence is presented herein that CUG expansions cause both a decrease in novel isoform mRNA levels and an imbalance of relative isoform mRNA levels in the cytoplasm. In its full length form, four domains have been identified in DMPK: an N-terminal leucine rich repeat, the serine-threonine kinase domain, an α-helical region presumably involved in protein-protein interactions, and a C-terminal domain (Brook et al., 1992; Fu et al., 1992; van der Ven et al., 1993). The C-terminal domain consists of a hydrophobic α-helix highly homologous to the transmembrane domains of HMG CoA reductase and rat microsomal aldehyde dehydrogenase, both known to be membrane anchored proteins (Liscum et al., 1985; Masaki et al., 1996). Cellular fractionation studies indicate that DMPK can be found both in cytosolic and membrane associated fractions (Maeda et al., 1995; Saitoh et al., 1996; Waring et al., 1996). In the novel isoform, the potential transmembrane domain found in exon 15 (Jansen et al., 1992) is eliminated due to the E14-E16 splice event. Also, the peptide encoded by E16 has a potential PKC phosphorylation site (FIG. 4), as determined by PROSITE, suggesting regulation of its activity by phosphorylation. While potential roles have been studied for DMPK in sodium channel regulation (Mounsey et al., 1995), membrane trafficking (Dunne et al., 1996; Dunne et al., 1996; Jin et al., 2000) and differentiation (Bush et al., 1996), no clear function has yet been established for any of its alternative isoforms. There is some evidence that DMPK forms multimers (Dunne et al., 1994; Waring et al., 1996); if so, an alteration in the relative levels of DMPK isoforms and the concomitant changes in the composition of DMPK complexes could clearly have dominant effects.

### References

Alwazzan, M., Newman, E., Hamshere, M. G., and Brook, J. D. (1999). Myotonic dystrophy is associated with a reduced level of RNA from the DMWD allele adjacent to the expanded repeat. Hum Mol Genet, 8, 1491-7.

Amack, J. D., Paguio, A. P., and Mahadevan, M. S. (1999). Cis and trans effects of the myotonic dystrophy (DM) mutation in a cell culture model. Hum Mol Genet, 8, 1975-84.

Barbaux, S., Niaudet, P., Gubler, M. C., Grunfeld, J. P., Jaubert, F., Kuttenn, F., Fekete, C. N., Souleyreau-Therville, N., Thibaud, E., Fellous, M., and McElreavey, K. (1997). Donor splice-site mutations in WT1 are responsible for Frasier syndrome. Nat Genet, 17, 467-70.

Berul, C. I., Maguire, C. T., Aronovitz, M. J., Greenwood, J., Miller, C., Gehrmann, J., Housman, D., Mendelsohn, M. E., and Reddy, S. (1999). DMPK dosage alterations result in atrioventricular conduction abnormalities in a mouse myotonic dystrophy model. J Clin Invest, 103, R1-7.

Bhagwati, S., Ghatpande, A., and Leung, B. (1996). Identification of two nuclear proteins which bind to RNA CUG repeats: significance for myotonic dystrophy. Biochem Biophys Res Commun, 228, 55-62.

Brook, J. D., McCurrach, M. E., Harley, H. G., Buckler, A. J., Church, D., Aburatani, H., Hunter, K., Stanton, V. P., Thirion, J. P., Hudson, T., and et al. (1992). Molecular basis of myotonic dystrophy: expansion of a trinucleotide (CTG) repeat at the 3′ end of a transcript encoding a protein kinase family member. Cell, 69, 385.

Bush, E. W., Taft, C. S., Meixell, G. E., and Perryman, M. B. (1996). Overexpression of myotonic dystrophy kinase in BC3H1 cells induces the skeletal muscle phenotype. J Biol Chem, 271, 548-52.

Carango, P., Noble, J. E., Marks, H. G., and Funanage, V. L. (1993). Absence of myotonic dystrophy protein kinase (DMPK) mRNA as a result of a triplet repeat expansion in myotonic dystrophy. Genomics, 18, 340-8.

Caskey, C. T., Swanson, M. S., and Timchenko, L. T. (1996). Myotonic dystrophy: discussion of molecular mechanism. Cold Spring Harb Symp Quant Biol, 61, 607-14.

Choi, Y. D., and Dreyfuss, G. (1984). Monoclonal antibody characterization of the C proteins of heterogeneous nuclear ribonucleoprotein complexes in vertebrate cells. J Cell Biol, 99, 1997-204.

Choi, Y. D., Grabowski, P. J., Sharp, P. A., and Dreyfuss, G. (1986). Heterogeneous nuclear ribonucleoproteins: role in RNA splicing. Science, 231, 1534-9.

Davis, B. M., McCurrach, M. E., Taneja, K. L., Singer, R. H., and Housman, D. E. (1997). Expansion of a CUG trinucleotide repeat in the 3′ untranslated region of myotonic dystrophy protein kinase transcripts results in nuclear retention of transcripts. Proc Natl Acad Sci USA, 94, 7388-93.

Dunne, P. W., Ma, L., Casey, D. L., and Epstein, H. F. (1996). Myotonic protein kinase expression in human and bovine lenses. Biochem Biophys Res Commun, 225, 281-8.

Dunne, P. W., Ma, L., Casey, D. L., Harati, Y., and Epstein, H. F. (1996). Localization of myotonic dystrophy protein kinase in skeletal muscle and its alteration with disease. Cell Motil Cytoskeleton, 33, 52-63.

Dunne, P. W., Walch, E. T., and Epstein, H. F. (1994). Phosphorylation reactions of recombinant human myotonic dystrophy protein kinase and their inhibition. Biochemistry, 33, 10809-14.

Fu, Y. H., Friedman, D. L., Richards, S., Pearlman, J. A., Gibbs, R. A., Pizzuti, A., Ashizawa, T., Perryman, M. B., Scarlato, G., Fenwick, R. G., Jr., and Caskey, C. T. (1993). Decreased expression of myotonin-protein kinase messenger RNA and protein in adult form of myotonic dystrophy [see comments]. Science, 260, 235-8.

Fu, Y. H., Pizzuti, A., Fenwick, R. G., Jr., King, J., Rajnarayan, S., Dunne, P. W., Dubel, J., Nasser, G. A., Ashizawa, T., de Jong, P., and et al. (1992). An unstable triplet repeat in a gene related to myotonic muscular dystrophy. Science, 255, 1256-8.

Garcia-Blanco, M. A., Jamison, S. F., and Sharp, P. A. (1989). Identification and purification of a 62,000-dalton protein that binds specifically to the polypyrimidine tract of introns. Genes Dev, 3, 1874-86.

Groenen, P., and Wieringa, B. (1998). Expanding complexity in myotonic dystrophy. Bioessays, 20, 901-12.

Hamshere, M. G., Newman, E. E., Alwazzan, M., Athwal, B. S., and Brook, J. D. (1997). Transcriptional abnormality in myotonic dystrophy affects DMPK but not neighboring genes. Proc Natl Acad Sci USA, 94, 7394-9.

Harper, P. S. (1989). Myotonic Dystrophy, 2nd Edition (Philadelphia: Saunders).

Hofmann-Radvanyi, H., Lavedan, C., Rabes, J. P., Savoy, D., Duros, C., Johnson, K., and Junien, C. (1993). Myotonic dystrophy: absence of CTG enlarged transcript in congenital forms, and low expression of the normal allele. Hum Mol Genet, 2, 1263-6.

Hunter, A., Tsilfidis, C., Mettler, G., Jacob, P., Mahadevan, M., Surh, L., and Korneluk, R. (1992). The correlation of age of onset with CTG trinucleotide repeat amplification in myotonic dystrophy. J Med Genet, 29, 774-9.

Jansen, G., Groenen, P. J., Bachner, D., Jap, P. H., Coerwinkel, M., Oerlemans, F., van den Broek, W., Gohlsch, B., Pette, D., Plomp, J. J., Molenaar, P. C., Nederhoff, M. G., van Echteld, C. J., Dekker, M., Berns, A., Hameister, H., and Wieringa, B. (1996). Abnormal myotonic dystrophy protein kinase levels produce only mild myopathy in mice [see comments]. Nat Genet, 13, 316-24.

Jansen, G., Mahadevan, M., Amemiya, C., Wormskamp, N., Segers, B., Hendriks, W., O'Hoy, K., Baird, S., Sabourin, L., Lennon, G., Jap, P. L., Iles, D., Coerwinkel, M., Hofker, M., Corrano, A. V., de Jong, P. J., Korneluk, R. G., and Wieringa, B. (1992). Characterization of the myotonic dystrophy region predicts multiple protein isoform-encoding mRNAs. Nat Genet, 1, 261-6.

Jin, S., Shimizu, M., Balasubramanyam, A., and Epstein, H. F. (2000). Myotonic dystrophy protein kinase (DMPK) induces actin cytoskeletal reorganization and apoptotic-like blebbing in lens cells. Cell Motil Cytoskeleton, 45, 133-148.

Kamma, H., Portman, D. S., and Dreyfuss, G. (1995). Cell type-specific expression of hnRNP proteins. Exp Cell Res, 221, 187-96.

Klamt, B., Koziell, A., Poulat, F., Wieacker, P., Scambler, P., Berta, P., and Gessler, M. (1998). Frasier syndrome is caused by defective alternative splicing of WT1 leading to an altered ratio of WT1+/−KTS splice isoforms. Hum Mol Genet, 7, 709-14.

Krahe, R., Ashizawa, T., Abbruzzese, C., Roeder, E., Carango, P., Giacanelli, M., Funanage, V. L., and Siciliano, M. J. (1995). Effect of myotonic dystrophy trinucleotide repeat expansion on DMPK transcription and processing. Genomics, 28, 1-14.

Liscum, L., Finer-Moore, J., Stroud, R. M., Luskey, K. L., Brown, M. S., and Goldstein, J. L. (1985). Domain structure of 3-hydroxy-3-methylglutaryl coenzyme A reductase, a glycoprotein of the endoplasmic reticulum. J Biol Chem, 260, 522-30.

Lou, H., Helfman, D. M., Gagel, R. F., and Berget, S. M. (1999). Polypyrimidine tract-binding protein positively regulates inclusion of an alternative 3′-terminal exon. Mol Cell Biol, 19, 78-85.

Maeda, M., Taft, C. S., Bush, E. W., Holder, E., Bailey, W. M., Neville, H., Perryman, M. B., and Bies, R. D. (1995). Identification, tissue-specific expression, and subcellular localization of the 80- and 71-kDa forms of myotonic dystrophy kinase protein. J Biol Chem, 270, 20246-9.

Mahadevan, M., Tsilfidis, C., Sabourin, L., Shutler, G., Amemiya, C., Jansen, G., Neville, C., Narang, M., Barcelo, J., O'Hoy, K., Leblond, S., Earle-MacDonald, J., de Jong, P., Wieringa, B., and Korneluk, R. G. (1992). Myotonic dystrophy mutation: an unstable CTG repeat in the 3′ untranslated region of the gene. Science, 255, 1253-5.

Masaki, R., Yamamoto, A., and Tashiro, Y. (1996). Membrane topology and retention of microsomal aldehyde dehydrogenase in the endoplasmic reticulum. J Biol Chem, 271, 16939-44.

Morrone, A., Pegoraro, E., Angelini, C., Zammarchi, E., Marconi, G., and Hoffman, E. P. (1997). RNA metabolism in myotonic dystrophy: patient muscle shows decreased insulin receptor RNA and protein consistent with abnormal insulin resistance. J Clin Invest, 99, 1691-8.

Mounsey, J. P., Xu, P., John, J. E. r., Home, L. T., Gilbert, J., Roses, A. D., and Moorman, J. R. (1995). Modulation of skeletal muscle sodium channels by human myotonin protein kinase. J Clin Invest, 95, 2379-84.

Patton, J. G., Porro, E. B., Galceran, J., Tempst, P., and Nadal-Ginard, B. (1993). Cloning and characterization of PSF, a novel pre-mRNA splicing factor. Genes Dev, 7, 393-406.

Philips, A. V., Timchenko, L. T., and Cooper, T. A. (1998). Disruption of splicing regulated by a CUG-binding protein in myotonic dystrophy [see comments]. Science, 280, 737-41.

Reddy, S., Smith, D. B., Rich, M. M., Leferovich, J. M., Reilly, P., Davis, B. M., Tran, K., Rayburn, H., Bronson, R., Cros, D., Balice-Gordon, R. J., and Housman, D. (1996). Mice lacking the myotonic dystrophy protein kinase develop a late onset progressive myopathy [see comments]. Nat Genet, 13, 325-35.

Redman, J. B., Fenwick, R. G., Jr., Fu, Y. H., Pizzuti, A., and Caskey, C. T. (1993). Relationship between parental trinucleotide GCT repeat length and severity of myotonic dystrophy in offspring. JAMA, 269, 1960-5.

Roberts, R., Timchenko, N. A., Miller, J. W., Reddy, S., Caskey, C. T., Swanson, M. S., and Timchenko, L. T. (1997). Altered phosphorylation and intracellular distribution of a (CUG)n triplet repeat RNA-binding protein in patients with myotonic dystrophy and in myotonin protein kinase knockout mice. Proc Natl Acad Sci USA, 94, 13221-6.

Sabourin, L. A., Mahadevan, M. S., Narang, M., Lee, D. S., Surh, L. C., and Korneluk, R. G. (1993). Effect of the myotonic dystrophy (DM) mutation on mRNA levels of the DM gene. Nat Genet, 4, 233-8.

Saitoh, N., Sasagawa, N., Koike, H., Shimokawa, M., Sorimachi, H., Ishiura, S., and Suzuki, K. (1996). Immunocytochemical localization of a full-length myotonin protein kinase in rat L6 myoblasts. Neurosci Lett, 218, 214-6.

Sarnat, H. B., and Silbert, S. W. (1976). Maturational arrest of fetal muscle in neonatal myotonic dystrophy. A pathologic study of four cases. Arch Neurol, 33, 466-74.

Swanson, M. S., and Dreyfuss, G. (1988). RNA binding specificity of hnRNP proteins: a subset bind to the 3′ end of introns. EMBO J, 7, 3519-29.

Taneja, K. L., McCurrach, M., Schalling, M., Housman, D., and Singer, R. H. (1995). Foci of trinucleotide repeat transcripts in nuclei of myotonic dystrophy cells and tissues. J Cell Biol, 128, 995-1002.

Timchenko, L. T., Miller, J. W., Timchenko, N. A., DeVore, D. R., Datar, K. V., Lin, L., Roberts, R., Caskey, C. T., and Swanson, M. S. (1996). Identification of a (CUG)n triplet repeat RNA-binding protein and its expression in myotonic dystrophy. Nucleic Acids Res, 24, 4407-14.

Timchenko, L. T., Timchenko, N. A., Caskey, C. T., and Roberts, R. (1996). Novel proteins with binding specificity for DNA CTG repeats and RNA CUG repeats: implications for myotonic dystrophy. Hum Mol Genet, 5, 115-21.

Tsilfidis, C., MacKenzie, A. E., Mettler, G., Barcelo, J., and Komeluk, R. G. (1992). Correlation between CTG trinucleotide repeat length and frequency of severe congenital myotonic dystrophy. Nat Genet, 1, 192-5.

van der Ven, P. F., Jansen, G., van Kuppevelt, T. H., Perryman, M. B., Lupa, M., Dunne, P. W., ter Laak, H. J., Jap, P. H., Veerkamp, J. H., Epstein, H. F., and et al. (1993). Myotonic dystrophy kinase is a component of neuromuscular junctions. Hum Mol Genet, 2, 1889-94.

Wang, J., Pegoraro, E., Menegazzo, E., Gennarelli, M., Hoop, R. C., Angelini, C., and Hoffman, E. P. (1995). Myotonic dystrophy: evidence for a possible dominant-negative RNA mutation. Hum Mol Genet, 4, 599-606.

Waring, J. D., Haq, R., Tamai, K., Sabourin, L. A., Ikeda, J. E., and Komeluk, R. G. (1996). Investigation of myotonic dystrophy kinase isoform translocation and membrane association. J Biol Chem, 271, 15187-93.

Zamore, P. D., and Green, M. R. (1989). Identification, purification, and biochemical characterization of U2 small nuclear ribonucleoprotein auxiliary factor. Proc Natl Acad Sci USA, 86, 9243-7.

Zhang, L., Liu, W., and Grabowski, P. J. (1999). Coordinate repression of a trio of neuron-specific splicing events by the splicing regulator PTB. RNA, 5, 117-30.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

What is claimed is:

1. An isolated and purified polypeptide or peptide which comprises an amino acid sequence encoded by exon 16 of the DMPK gene.

2. The isolated and purified polypeptide or peptide of claim 1 which comprises SEQ ID NO:2.

3. The isolated and purified polypeptide or peptide of claim 1 which is a fusion polypeptide or peptide.

4. An isolated and purified nucleic acid molecule which encodes the polypeptide or peptide of claim 1.

5. The isolated and purified nucleic acid molecule of claim 4 which is RNA.

6. The isolated and purified nucleic acid molecule of claim 4 which is cDNA.

7. The isolated and purified nucleic acid molecule of claim 5 which binds hnRNP C, U2AF, PTB, PSF, or any combination thereof.

8. An antibody which specifically binds the polypeptide or peptide of claim 1.

9. A fusion polypeptide comprising SEQ ID NO:2.

10. A fusion polypeptide comprising an amino acid sequence encoded by exons 9-15 of the DMPK gene.

11. A method to detect an isoform of DMPK, comprising:
a) contacting a sample of nucleic acid obtained from a biological sample with at least one primer specific for an isoform of DMPK RNA which includes exon 16 under conditions effective to amplify at least a portion of DMPK nucleic acid so as to yield amplified DMPK nucleic acid; and
b) detecting or determining the presence, absence or amount of the isoform.

12. The method of claim 11 wherein the sample is contacted with at least one primer specific for at least one other isoform of DMPK other than that which includes exon 16 under conditions effective to amplify DMPK nucleic acid so as to yield amplified DMPK nucleic acid and detecting or determining the presence, absence or amount of the isoform of DMPK that is not the isoform which includes exon 16.

13. A method to detect an isoform of DMPK, comprising:
a) contacting a biological sample from a mammal with the antibody of claim 8 so as to form a complex; and
b) detecting or determining complex formation.

14. The method of claim 13 wherein the mammal has, or is at risk of, myotonic dystrophy.

15. The method of claim 13 wherein the mammal has, or is at risk of, a myocardial infarction.

16. The method of claim 13 wherein the mammal has, or is at risk of, a condition associated with muscle damage.

17. The method of claim 13 wherein the sample is contacted with an antibody that specifically binds to isoforms of DMPK other than one which is encoded by RNA comprising DMPK exon 16.

18. A diagnostic method comprising: detecting or determining in a physiological sample from a mammal at risk of, or having, a condition associated with muscle damage or dysfunction the amount of a DMPK RNA comprising DMPK exon 16 and correlating the amount to muscle damage or dysfunction in the mammal.

19. A diagnostic method comprising: detecting or determining in a physiological sample from a mammal at risk of, or having, a condition associated with muscle damage or dysfunction the relative amount of RNA encoding at least two isoforms of DMPK and correlating the amount to muscle damage or dysfunction in the mammal, wherein at least one DMPK isoform is encoded by RNA comprising DMPK exon 16.

20. A diagnostic method comprising: detecting or determining in a physiological sample from a mammal at risk of, or having, a condition associated with muscle damage or dysfunction the amount of a DMPK isoform encoded by RNA comprising DMPK exon 16 and correlating the amount to muscle damage or dysfunction in the mammal.

21. A diagnostic method comprising: detecting or determining in a physiological sample from a mammal at risk of, or having, a condition associated with muscle damage or dysfunction the relative amount of at least two DMPK isoforms and correlating the amount to muscle damage or dysfunction in the mammal, wherein at least one DMPK isoform is encoded by RNA comprising DMPK exon 16.

22. The method of claim 18, 19, 20 or 21 wherein the condition is myotonic dystrophy.

23. The method of claim 18, 19, 20 or 21 wherein the condition is myocardial infarction.

24. The method of claim 20 or 21 wherein the physiological sample is a physiological fluid sample.

25. The method of claim 24 wherein the sample is a blood sample.

26. The method of claim 25 wherein the sample is a serum sample.

27. A diagnostic kit for detecting or determining a DMPK isoform which comprises packaging, containing, separately packaged,
a) a solid phase which binds a capture antibody; and
b) a known amount of a detection antibody, wherein the detection antibody comprises the antibody of claim 8.

28. A diagnostic kit for detecting or determining the ratio of DMPK isoforms which comprises packaging, containing, separately packaged,
a) the antibody of claim 8; and
b) an antibody which detects isoforms of DMPK other than one encoded by nucleic acid comprising exon E16.

29. The kit of claim 27 or 28 further comprising an antibody which detects at least one isoform of DMPK other than one encoded by RNA comprising exon E16.

30. The kit of claim 27 wherein the detection antibody is detectably labeled.

31. The antibody of claim 8 which specifically binds to SEQ ID NO:2 or a portion thereof.

32. The method of claim 11 further comprising contacting a second sample with at least one primer specific for at least one other isoform of DMPK other than that which includes exon 16 under conditions effective to amplify DMPK nucleic acid so as to yield amplified DMPK nucleic acid and detecting or determining the presence, absence or amount of the isoform of DMPK that is not the isoform which includes exon 16.

33. The method of claim 13 further comprising contacting a second sample with an antibody that specifically binds to isoforms of DMPK other than one which is encoded by RNA comprising DMPK exon 16.

34. The isolated and purified polypeptide or peptide of claim 1 which comprises SEQ ID NO:14.

35. The isolated and purified polypeptide or peptide of claim 1 which comprises SEQ ID NO:15.

36. The method of claim 18 wherein detecting or determining the amount of the DMPK RNA comprising DMPK exon 16 comprises the use of SEQ ID NO:16, SEQ ID NO:17, or SEQ ID NO:18.

37. An immunoconjugate comprising SEQ ID NO:14 and a carrier.

38. The immunoconjugate of claim 37 wherein the carrier is KLH.

39. An immunoconjugate comprising SEQ ID NO:15 and a carrier.

40. The immunoconjugate of claim 39 wherein the carrier is KLH.


SEQUENCE LISTING (22 sequences)

SEQ ID NO: 1 - DNA, 129 bp, Homo sapiens
gctgaggccctgacgtggatgggcaaactgcaggcctgggaaggcagcaagccgggccgtccgtgttccatcctccacgcacccccacctatcgttggttcgcaaagtgcaaagctttcttgtgcatga

SEQ ID NO: 2 - protein, 42 aa, Homo sapiens
AEALTWMGKLQAWEGSKPGRPCSILHAPPPIVGSQSAKLSCA

SEQ ID NO: 3 - DNA, 22 bp, Homo sapiens
actggagctgggcggagaccca

SEQ ID NO: 4 - DNA, 18 bp, Homo sapiens
gggcagatggagggcctt

SEQ ID NO: 5 - DNA, 18 bp, Homo sapiens
gctgaagtggcagttcca

SEQ ID NO: 6 - DNA, 22 bp, Homo sapiens
tgtcggggtctcagtgcatcca

SEQ ID NO: 7 - DNA, 23 bp, Homo sapiens
ctcttcgcatcgctgtctgcgag

SEQ ID NO: 8 - DNA, 19 bp, Homo sapiens
gtcgggggtgggggtccat

SEQ ID NO: 9 - DNA, 20 bp, Homo sapiens
ggggtccacctgccttttgt

SEQ ID NO: 10 - DNA, 19 bp, Homo sapiens
gagtcgaagacagttctag

SEQ ID NO: 11 - DNA, 20 bp, Homo sapiens
gggagacactgtcggacatt

SEQ ID NO: 12 - DNA, 19 bp, Homo sapiens
acgtcagggcctcagccct

SEQ ID NO: 13 - DNA, 15 bp, Mus musculus
ctgctgcagcagctg

SEQ ID NO: 14 - protein, 22 aa, Homo sapiens
AEALTWMGKLQAWEGSKPGRPC

SEQ ID NO: 15 - protein, 20 aa, Homo sapiens
SILHAPPPIVGSQSAKLSCA

SEQ ID NO: 16 - RNA, 166 nt, Artificial Sequence
  Antisense RNA.
cccacuuugcgaaccaacgauaggugggggugcguggaggauggaacacggacggcccggcuugcugccuucccaggccugcaguuugcccauccacgucagggccucagccuggccgaaagaaagaaauggucugugauccccccgcuauaggcugaaccacgau

SEQ ID NO: 17 - RNA, 285 nt, Artificial Sequence
  Antisense RNA.
gaggaggggggcgcgggauccccgaaaaagcggguuuggcaaaagcaaauuucccgaguaagcaggcagagaucgcgccacacgcuccccagagcagggcgucaugcacaagaaagcuuugcacuuugcgaaccaacgauaggugggggugcgcggaggauggaacacggacggcccggcuugcugccuucccaggccugcaguuugcccauccacgucagggccucagccuggccgaaagaaagaaauggucugugauccccccgcuauaggcugaaccacgau

SEQ ID NO: 18 - RNA, 301 nt, Artificial Sequence
  Antisense RNA.
gaggaggggggcgcgggauccccgaaaaagcggguuuggcaaaagcaaauuucccgaguaagcaggcagagaucgcgccacacgcuccccagagcagggcgucaugcacaagaaagcuuugcacuuugcgaaccaacgauaggugggggugcgcggaggauggaacacggacggcccggcuugcugccuucccaggccugcaguuugcccauccacgucagggccucagcccuggcagggagcagcagguggcggcggugcauggggccuggccccaccagauauagucaacgucgcguac

SEQ ID NO: 19 - DNA, 894 bp, Homo sapiens
ctgctgctccctgccagggtccctaggcctggcctatcggaggcgctttccctgctcctgttcgccgttgttctgtctcgtgccgccgccctgggctgcattgggttggtggcccacgccggccaactcaccgcagtctggcgccgcccaggagccgcccgcgctccctgaaccctagaactgtcttcgactccggggccccgttggaagactgagtgcccggggcacggcacagaagccgcgcccaccgcctgccagttcacaaccgctccgagcgtgggtctccgcccagctccagtcctgtgtaccgggcccgccccctagcggccggggagggaggggccgggtccgcggccggcgaacggggctcgaagggtccttgtagccgggaatgctgctgctgctgctggggggatcacagaccatttctttctttcggccaggctgaggccctgacgtggatgggcaaactgcaggcctgggaaggcagcaagccgggccgtccgtgttccatcctccacgcacccccacctatcgttggttcgcaaagtgcaaagctttcttgtgcatgacgccctgctctggggagcgtctggcgcgatctctgcctgcttactcgggaaatttgcttttgccaaacccgctttttcggggatcccgcgcccccctcctcacttgcgctgctctcggagccccagccggctccgcccgcttcggcggtttggatatttattgacctcgtcctccgactcgctgacaggctacaggacccccaacaaccccaatccacgttttggatgcactgagaccccgacattcctcggtatttattgtctgtccccacctaggacccccacccccgaccctcgcgaataaaaggccctccatctgccc

SEQ ID NO: 20 - protein, 56 aa, Homo sapiens
LLLPARVPRPGLSEALSLLLFAVVLSRAAALGCIGLVAHAGQLTAVWRRPGAARAP

SEQ ID NO: 21 - DNA, 23 bp, Homo sapiens
aatgctgctgctgctgctggggg

SEQ ID NO: 22 - DNA, 19 bp, Artificial Sequence
  A mutated DMPK 3′UTR fragment.
gatcctgcagctctggggg